(19) United States
(12) Patent Application Publication
(10) Pub. No.: US 2010/0121013 A1
(43) Pub. Date: May 13, 2010

(54) CURABLE COMPOSITION FOR PHOTOIMPRINT, ITS CURED PRODUCT AND PRODUCTION METHOD FOR IT, AND COMPONENT OF LIQUID-CRYSTAL DISPLAY DEVICE

(75) Inventors: Kyouhei SAKITA, Shizuoka (JP); Hiroyuki Yonezawa, Shizuoka (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/613,323

(22) Filed: Nov. 5, 2009

(30) Foreign Application Priority Data
Nov. 7, 2008 (JP) 2008-286069

(51) Int. Cl. C08F 222/26; C07C 69/527; B29C 59/16

(52) U.S. Cl. 526/323; 560/225; 264/447

(57) ABSTRACT

A curable composition for photoimprints, comprising a (meth)acrylate compound represented by the following formula (1):

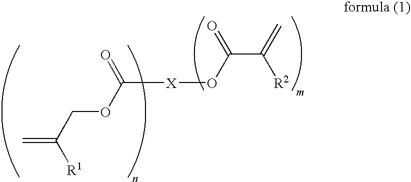

wherein R¹ and R² represent a hydrogen atom or a methyl group; X represents a single bond or an aliphatic group; n and m indicate an integer of 1 or more, has a low viscosity and a high transfer patterning accuracy and gives a cured film excellent in scratch resistance and adhesiveness.

### BACKGROUND OF THE INVENTION

1. Field of the Invention

The present invention relates to a curable composition for use for photoimprints, its cured product and a production method for it, and to a component comprising the cured product for liquid-crystal display devices.

2. Description of the Related Art

Two methods of nanoimprinting technology have been proposed; one comprises using a thermoplastic resin as the material to be processed (see S. Chou et al., Appl. Phys. Lett., Vol. 67, 3114 (1995)), and the other comprises using a curable composition for photonanoimprint lithography (see M. Colbun et al., Proc. SPIE, Vol. 3676, 379 (1999)). In a thermal nanoimprinting method using a thermoplastic resin, a polymer resin heated up to a temperature not lower than the glass transition temperature thereof is pressed against a mold, then cooled, and thereafter the mold is released to thereby transfer a microstructure onto the resin on a substrate. This method is applicable to various resin materials and glass materials, and is therefore expected to be applied to various fields. For example, U.S. Pat. No. 5,772,905 and U.S. Pat. No. 5,956,216 disclose a nanoimprinting method of using a thermoplastic resin to form nanopatterns at a low cost.

On the other hand, in a photonanoimprinting method of photocuring a curable composition for nanoimprints by irradiation with light through a transparent mold, imprinting at room temperature is possible. Recently, novel development of a nanocasting method that takes the advantages of the two as combined and a reversal imprinting method of producing a three-dimensional laminate structure has been reported.

In such nanoimprinting methods, the following applied technologies have been proposed. The first technology is to apply the method to production of high-density semiconductor integrated circuits and production of transistors for liquid-crystal displays in place of conventional lithography, taking the advantages of the high-level positioning accuracy in the method and the high-level integration scale by the method. The second technology is application of formed patterns having various functions to various nanotechnology element components or to various structural components; and its examples include various micro/nano optical elements and high-density recording media, as well as structural components of optical films and flat panel displays, etc. In still other technologies, simultaneous integral formation of a microstructure and a nanostructure, or construction of a laminate structure through simple interlayer positioning is applied to production of μ-TAS or biochips. Including the above-mentioned technologies, approaches to practical use of the nanoimprinting methods relating to those applications have been activated these days.

First described are cases of application of the above-mentioned first technology to production of high-density semiconductor integrated circuits. Recently, microstructuring and integration up-scaling for semiconductor integrated circuits are being much advanced; and as a pattern transfer technology for realizing the microfabrication, advanced high-accuracy photolithography is promoted. On the other hand, use of a nanoimprint lithography technology that has been proposed as a technology for micropatterning at a low cost (photonanoimprinting method) is investigated. For example, a nanoimprinting technology is known, in which a silicon wafer is used as a stamper to produce a microstructure of at most 25 nm in size by transferring.

Next described are cases of application of nanoimprint lithography of the above-mentioned second technology to flat displays such as liquid-crystal displays (LCD), plasma display panels (PDP), etc. With the recent tendency toward large-sized LCD substrates and PDP substrates for high-definition microprocessing thereon, photonanoimprint lithography has become specifically noted these days as an inexpensive lithography technology capable of being substituted for conventional photolithography for use in production of thin-film transistors (TFT) and electrode plates. Accordingly, it has become necessary to develop a photocurable resist capable of being substituted for the etching photoresist for use in conventional photolithography. In addition, application of photonanoimprinting lithography is being investigated to transparent protective materials for use as structural components of LCD and to spacers that define the cell gap in liquid-crystal displays. Differing from the above-mentioned etching resist, the resist for such structural components finally remains in displays, and therefore, it may be referred to as “permanent resist” or “permanent film”.

The permanent film to which conventional photography is applied includes, for example, a protective film to be provided on the TFT substrate of a liquid-crystal panel, and a protective film to be provided on a color filter for reducing the step difference between red (R), green (G) and blue (B) layers and for securing the resistance to high-temperature treatment in sputtering for ITO film formation.

In the field of spacers for use in liquid-crystal displays, in general, a photocurable composition comprising a resin, a photopolymerizable monomer and a photopolymerization initiator is widely used in conventional photolithography. In general, the spacer is formed after formation of a color filter or after formation of a color filter protective film, by applying a photocurable composition onto the color filter substrate through photolithography to form thereon a pattern having a size of from 10 μm to 20 μm or so, followed by heating and curing it by post-baking.

The problem characteristic of the curable composition for photonanoimprints is that the composition must secure resist flowability into the concave part of a mold and must have a low viscosity in the absence of a solvent or in the presence of a small amount of a solvent, and that the pattern accuracy must be taken into consideration for the composition. Further, the composition must satisfy other various requirements in accordance with the use thereof. For example, the composition must satisfy scratch resistance of the formed film and adhesiveness thereof.

### SUMMARY OF THE INVENTION

The present invention has been made in consideration of the above-mentioned situation, and its object is to provide a curable photoimprint composition excellent in photocurability, especially a curable photoimprint composition favorable for permanent films in flat panel displays, etc. Concretely, the invention is to provide a curable composition for photoimprints which has a low viscosity and ensures high transfer patterning accuracy and which is excellent in scratch resistance and adhesiveness of the formed film.

Given the situation as above, the present inventors have assiduously studied and, as a result, have found that use of an allyl ester group-having (meth)acrylate compound can solve the above-mentioned problems. Concretely, the inventors have solved the problems according to the following means:

(1) A curable composition for photoimprints, comprising a (meth)acrylate compound represented by the following formula (1):

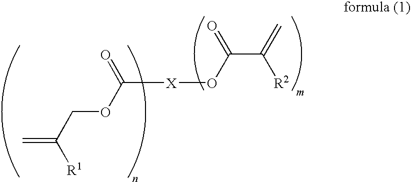

wherein R¹ and R² each represent a hydrogen atom or a methyl group; X represents a single bond or an aliphatic group; n and m each indicate an integer of 1 or more; when m or n is 2 or more, the respective R¹'s or R²'s may be the same or different.

(2) The curable composition for photoimprints of (1), wherein X in formula (1) is an aliphatic group having from 2 to 4 carbon atoms.

(3) The curable composition for photoimprints of (1) or (2), wherein n+m in formula (1) is an integer of from 3 to 5.

(4) The curable composition for photoimprints of (1) or (2), wherein n and m in formula (1) each are an integer of from 1 to 3.

(5) The curable composition for photoimprints of any one of (1) to (4), wherein the viscosity at 25° C. of the (meth)acrylate compound of formula (1) is at most 100 mPa·s.

(6) The curable composition for photoimprints of anyone of (1) to (5), which further comprises a silicon compound.

(7) The curable composition for photoimprints of any one of (1) to (6), which further comprises an antioxidant.

(8) A compound represented by the following formula (2), (3) or (4):

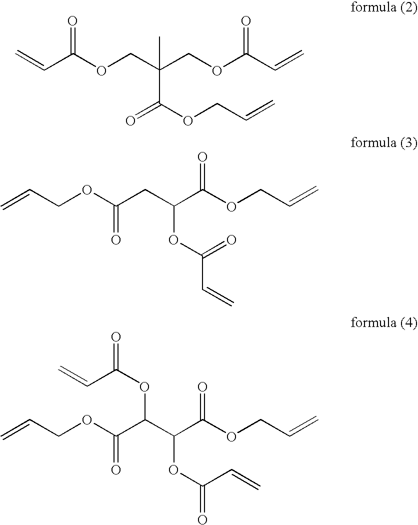

(9) A cured product produced by curing the curable composition for photoimprints of any one of (1) to (7).

(10) A component of liquid-crystal display devices, comprising the cured product of (9).

(11) A method for producing a cured product, comprising:

applying a curable composition for photoimprints of any one of (1) to (7) onto a substrate and patterning it thereon,

pressing a mold against the patterned layer, and

photoirradiating the patterned layer.

(12) The method for producing a cured product of (11), which further includes heating the photoirradiated patterned layer.

The invention has made it possible to provide a curable composition for nanoimprint lithography which has a low viscosity and secures high transfer patterning accuracy and which is excellent in scratch resistance and adhesiveness of the formed film.

### BEST MODE FOR CARRYING OUT THE INVENTION

The contents of the invention are described in detail hereinunder. In this specification, the numerical range expressed by the wording “a number to another number” means the range that falls between the former number indicating the lowermost limit of the range and the latter number indicating the uppermost limit thereof. In this specification, mass ratio is equal to weight ratio.

In this specification, “(meth)acrylate” means acrylate and methacrylate; “(meth)acrylic” means acrylic and methacrylic; “(meth)acryloyl” means acryloyl and methacryloyl. In the invention, monomer is differentiated from oligomer and polymer, and the monomer indicates a compound having a weight-average molecular weight of at most 1,000. In this specification, “functional group” means a group participating in polymerization.

“Imprint” referred to in the invention is meant to indicate pattern transfer in a size of from 1 nm to 10 mm and preferably meant to indicate pattern transfer in a size of from about 10 nm to 100 μm (nanoimprint).

The curable composition for photoimprints of the invention (hereinafter this may be simply referred to as “the composition of the invention”) has a low viscosity before cured, and is excellent in micropatternability. After cured, the composition gives a cured film excellent in surface hardness and scratch resistance and totally further excellent in other various physical properties of coating film. Accordingly, the composition of the invention is widely usable in photonanoimprint lithography.

Specifically, the composition of the invention exhibits the following characteristics when used in photonanoimprint lithography.

(1) As excellent in solution flowability at room temperature, the composition can readily flow into the cavity of the concave part of a mold and hardly takes air therein to cause bubble defects; and after photocured, the composition leaves few residues in both the concave part and the convex part of a mold.

(2) After cured, the cured film is excellent in patterning accuracy and in mechanical properties such as surface hardness and scratch resistance. In addition, the cured film is excellent in adhesiveness between the coating film and the substrate and also in releasability of the coating film from a mold. Therefore, when the cured film is peeled away from a mold, it is free from troubles of pattern deformation or coating film surface stringiness to cause surface roughness, and the cured film may form a good pattern.

(3) As excellent in coating uniformity, the composition is suitable to the filed of application to large-size substrates and micropatterning thereon.

For example, the composition of the invention is favorably applied to semiconductor integrated circuits and components of liquid-crystal display devices (especially to microfabrication for thin-film transistors of liquid-crystal displays, protective films of liquid-crystal color filters, spacers and other components of liquid-crystal display devices, etc.) to which, however, conventional compositions are heretofore difficult to apply; and in addition, the composition of the invention is further applicable to production of any others, widely for example, partitioning materials for plasma display panels, flat screens, microelectromechanical systems (MEMS), sensor devices, optical discs, magnetic-recording media such as high-density memory discs, optical parts such as diffraction gratings and relief holograms, nanodevices, optical devices, optical films, polarization devices, organic transistors, color filters, overcoat layers, pillar materials, rib materials for liquid-crystal alignment, microlens arrays, immunoassay chips, DNA separation chips, microreactors, nanobio devices, optical waveguides, optical filters, photonic liquid crystals, etc.

### (Polymerizable Monomer)

The composition of the invention contains a (meth)acrylate compound of formula (1) as a polymerizable monomer for the purpose of increasing the hardness and enhancing the scratch resistance of the cured product thereof.

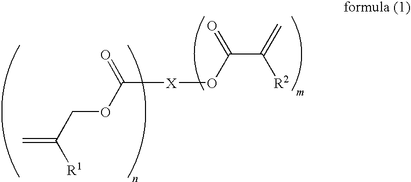

wherein R¹ and R² each represent a hydrogen atom or a methyl group; X represents a single bond or an aliphatic group; n and m each indicate an integer of 1 or more; when m or n is 2 or more, the respective R¹'s or R²'s may be the same or different.

Preferably, R¹ and R² each are a hydrogen atom, more preferably they are both hydrogen atoms from the viewpoint of the viscosity and the curability of the composition.

Preferably, m and n each are an integer of from 1 to 3, more preferably 1 or 2. Preferably, n+m is an integer of from 2 to 6, more preferably an integer of from 3 to 5, even more preferably 3 or 4.

X is preferably an aliphatic group, more preferably an aliphatic group having from 2 to 4 carbon atoms, even more preferably an aliphatic group having 3 or 4 carbon atoms. The aliphatic group is preferably an alkylene group or an alkenylene group, more preferably an alkylene group. The alkylene group may have a substituent, and the substituent is preferably an alkyl group.

For expressing high hardness and good scratch resistance after cured, the amount of the (meth)acrylate compound of formula (1) in the composition of the invention is preferably at least 20% by mass, more preferably at least 22% by mass, even more preferably at least 24% by mass. The uppermost limit of the amount is not specifically defined, and in general, it may be at most 90% by mass.

In general, the molecular weight of the (meth)acrylate compound of formula (1) is preferably at most 600, more preferably at most 500, even more preferably at most 400 for realizing the low viscosity level of the composition of the invention. Not specifically defined, the lowermost limit of the molecular weight may be generally at least 170, preferably at least 200 from the viewpoint of the volatility of the compound.

Preferably, the viscosity at 25° C. of the (meth)acrylate compound of formula (1) is at most 100 mPa·s.

Examples of the (meth)acrylate compound of formula (1) include the following:

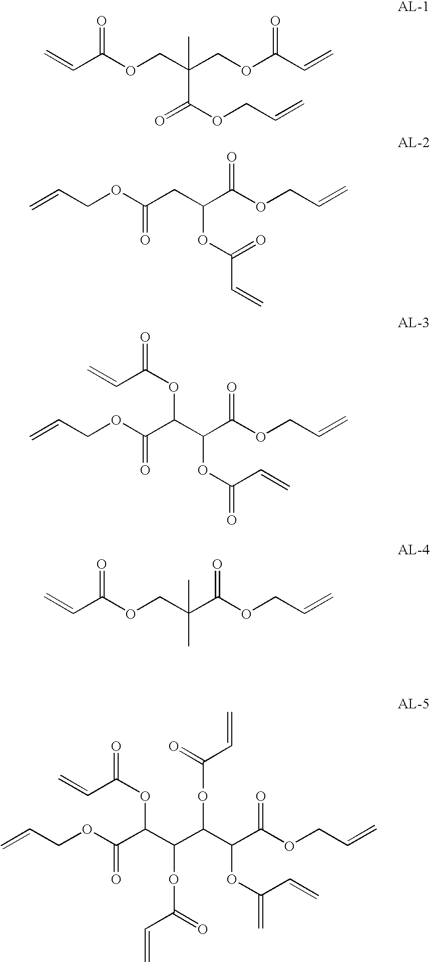

In addition to the (meth)acrylate compound represented by the formula (1), the composition of the invention may comprise other polymerizable monomers. As the other polymerizable monomer, the composition may comprise a polymerizable unsaturated monomer having one ethylenic unsaturated bond-containing group (monofunctional polymerizable unsaturated monomer). The viscosity of the composition can be further reduced by adding the other polymerizable monomer to the composition. Examples of the other polymerizable monomer include 2-acryloyloxyethyl phthalate, 2-acryloyloxy-2-hydroxyethyl phthalate, 2-acryloyloxyethyl hexahydrophthalate, 2-acryloyloxypropyl phthalate, 2-ethyl-2-butylpropanediol acrylate, 2-ethylhexyl (meth)acrylate, 2-ethylhexylcarbitol (meth)acrylate, 2-hydroxybutyl (meth)acrylate, 2-hydroxyethyl (meth)acrylate, 2-hydroxypropyl (meth)acrylate, 2-methoxyethyl (meth)acrylate, 3-methoxybutyl (meth)acrylate, 4-hydroxybutyl (meth)acrylate, acrylic acid dimer, benzyl (meth)acrylate, butanediol mono(meth)acrylate, butoxyethyl (meth)acrylate, butyl (meth)acrylate, cetyl (meth)acrylate, ethyleneoxide-modified (hereinafter this may be referred to as “EO”) cresol (meth)acrylate, dipropylene glycol (meth)acrylate, γ-(meth)acryloxypropyltrimethoxysilane, ethoxylated phenyl (meth)acrylate, ethyl (meth)acrylate, isoamyl (meth)acrylate, isobutyl (meth)acrylate, isooctyl (meth)acrylate, cyclohexyl (meth)acrylate, isobornyl (meth)acrylate, dicyclopentanyl (meth)acrylate, dicyclopentanyloxyethyl (meth)acrylate, isomyristyl (meth)acrylate, lauryl (meth)acrylate, methoxydiproylene glycol (meth)acrylate, methoxytripropylene glycol (meth)acrylate, methoxypolyethylene glycol (meth)acrylate, methoxytriethylene glycol (meth)acrylate, methyl (meth)acrylate, neopentyl glycol benzoate (meth)acrylate, nonylphenoxypolyethylene glycol (meth)acrylate, nonylphenoxypolypropylene glycol (meth)acrylate, octyl (meth)acrylate, paracumylphenoxyethylene glycol (meth)acrylate, epichlorohydrin (hereinafter referred to as “ECH”)-modified phenoxyacrylate, phenoxyethyl (meth)acrylate, phenoxydiethylene glycol (meth)acrylate, phenoxyhexaethylene glycol (meth)acrylate, phenoxytetraethylene glycol (meth)acrylate, polyethylene glycol (meth)acrylate, polyethylene glycol-polypropylene glycol (meth)acrylate, polypropylene glycol (meth)acrylate, stearyl (meth)acrylate, EO-modified succinic acid (meth)acrylate, tert-butyl (meth)acrylate, tribromophenyl (meth)acrylate, EO-modified tribromophenyl (meth)acrylate, tridodecyl (meth)acrylate, p-isopropenylphenol, styrene, α-methylstyrene, acrylonitrile and vinyl carbazole.

As the other polymerizable monomer, also preferred is a polyfunctional polymerizable unsaturated monomer having two or more ethylenic unsaturated bond-containing groups.

Preferred examples of the difunctional polymerizable unsaturated monomer having two ethylenic unsaturated bond-containing groups for use in the invention include diethylene glycol monoethyl ether (meth)acrylate, dimethylol-dicyclopentane di(meth)acrylate, di(meth)acrylated isocyanurate, 1,3-butylene glycol di(meth)acrylate, 1,4-butanediol di(meth)acrylate, EO-modified 1,6-hexanediol di(meth)acrylate, ECH-modified 1,6-hexanediol di(meth)acrylate, allyloxy-polyethylene glycol acrylate, 1,9-nonanediol di(meth)acrylate, EO-modified bisphenol A di(meth)acrylate PO-modified bisphenol A di(meth)acrylate, modified bisphenol A di(meth)acrylate, EO-modified bisphenol F di(meth)acrylate, ECH-modified hexahydrophthalic acid diacrylate, hydroxypivalic acid neopentyl glycol di(meth)acrylate, neopentyl glycol di(meth)acrylate, EO-modified neopentyl glycol diacrylate, propyleneoxide (hereinafter referred to as “PO”)-modified neopentyl glycol diacrylate, caprolactone-modified hydroxypivalate neopentyl glycol, stearic acid-modified pentaerythritol di(meth)acrylate, ECH-modified phthalic acid di(meth)acrylate, poly(ethylene glycol-tetramethylene glycol) di(meth)acrylate, poly(propylene glycol-tetramethylene glycol) di(meth)acrylate, polyester (di)acrylate, polyethylene glycol di(meth)acrylate, polypropylene glycol di(meth)acrylate, ECH-modified propylene glycol di(meth)acrylate, silicone di(meth)acrylate, triethylene glycol di(meth)acrylate, tetraethylene glycol di(meth)acrylate, dimethyloltricyclodecane di(meth)acrylate, neopentyl glycol-modified trimethylolpropane di(meth)acrylate, tripropylene glycol di(meth)acrylate, EO-modified tripropylene glycol di(meth)acrylate, triglycerol di(meth)acrylate, dipropylene glycol di(meth)acrylate, divinylethylene-urea, divinylpropylene-urea.

Of those, especially preferred for use in the invention are neopentyl glycol di(meth)acrylate, 1,4-butanediol di(meth)acrylate, 1,9-nonanediol di(meth)acrylate, tripropylene glycol di(meth)acrylate, tetraethylene glycol di(meth)acrylate, hydroxypivalate neopentyl glycol di(meth)acrylate, polyethylene glycol di(meth)acrylate, etc.

The composition of the invention may comprise a polyfunctional oligomer or polymer having a molecular weight higher than the above polyfunctional monomer as the other polymerizable monomer to further increase the crosslink density, not detracting from the object of the invention. Examples of photopolymerizable polyfunctional oligomer and photopolymerizable polyfunctional polymer include polyester acrylate, ester acrylate oligomer, polyurethane acrylate, urethane acrylate oligomer, polyether acrylate, ether acrylate oligomer, polyepoxy acrylate and epoxy acrylate oligomer.

As the other polymerizable monomer used in the invention, oxirane ring-having compounds can be used. Examples of the oxirane ring-having compound include polyglycidyl esters of polybasic acids, polyglycidyl ethers of polyalcohols, polyglycidyl ethers of polyoxyalkylene glycols, polyglycidyl ethers of aromatic polyols, hydrogenated polyglycidyl ethers of aromatic polyols, urethane-polyepoxy compounds, epoxidated polybutadienes, etc. One or more of these compounds may be used either singly or as combined.

Examples of the oxirane ring-having compound preferred for use in the invention include bisphenol A diglycidyl ether, bisphenol F diglycidyl ether, bisphenol S diglycidyl ether, brominated bisphenol A diglycidyl ether, brominated bisphenol F diglycidyl ether, brominated bisphenol S diglycidyl ether, hydrogenated bisphenol A diglycidyl ether, hydrogenated bisphenol F diglycidyl ether, hydrogenated bisphenol S diglycidyl ether, 1,4-butanediol diglycidyl ether, 1,6-hexanediol diglycidyl ether, glycerin triglycidyl ether, trimethylolpropane triglycidyl ether, polyethylene glycol diglycidyl ether, polypropylene glycol diglycidyl ether; polyglycidyl ethers of polyether polyols produced by adding one or more alkylene oxides to aliphatic polyalcohol such as ethylene glycol, propylene glycol, glycerin or the like; diglycidyl esters of aliphatic long-chain dibasic acids; monoglycidyl ethers of aliphatic higher alcohols; monoglycidyl ethers of polyether alcohols produced by adding alkyleneoxide to phenol, cresol, butylphenol or the like; glycidyl esters of higher fatty acids, etc.

Of those, especially preferred are bisphenol A diglycidyl ether, bisphenol F diglycidyl ether, hydrogenated bisphenol A diglycidyl ether, hydrogenated bisphenol F diglycidyl ether, 1,4-butanediol diglycidyl ether, 1,6-hexanediol diglycidyl ether, glycerin triglycidyl ether, trimethylolpropane triglycidyl ether, neopentyl glycol diglycidyl ether, polyethylene glycol diglycidyl ether, polypropylene glycol diglycidyl ether.

Commercial products favorable for use herein as the glycidyl group-having compound are UVR-6216 (by Union Carbide), Glycidol, AOEX24, Cyclomer A200 (all by Daicel Chemical Industry), Epikote 828, Epikote 812, Epikote 1031, Epikote 872, EpikoteCT508 (all by Yuka Shell), KRM-2400, KRM-2410, KRM-2408, KRM-2490, KRM-2720, KRM-2750 (all by Asahi Denka Kogyo), etc. One or more of these may be used either singly or as combined.

The production method for the oxirane ring-having compounds is not specifically defined. For example, the compounds may be produced with reference to publications of Lecture of Experimental Chemistry 20, 4th Ed., Organic Synthesis II, p. 213, ff. (Maruzen, 1992); The chemistry of heterocyclic compounds—Small Ring Heterocycles, Part 3, Oxiranes (edited by Alfred Hasfner, John & Wiley and Sons, An Interscience Publication, New York, 1985); Yoshimura, Adhesive, Vol. 29, No. 12, 32, 1985; Yoshimura, Adhesive, Vol. 30, No. 5, 42, 1986; Yoshimura, Adhesive, Vol. 30, No. 7, 42, 1986; JP-A-11-100378, Japanese Patents 2906245 and 2926262.

As the other polymerizable monomer for use in the invention, vinyl ether compounds may be in the composition.

Any known vinyl ether compounds are usable, including, for example, 2-ethylhexyl vinyl ether, butanediol 1,4-divinyl ether, diethylene glycol monovinyl ether, ethylene glycol divinyl ether, triethylene glycol divinyl ether, 1,2-propanediol divinyl ether, 1,3-propanediol divinyl ether, 1,3-butanediol divinyl ether, 1,4-butanediol divinyl ether, tetramethylene glycol divinyl ether, neopentyl glycol divinyl ether, trimethylolpropane trivinyl ether, trimethylolethane trivinyl ether, hexanediol divinyl ether, tetraethylene glycol divinyl ether, pentaerythritol divinyl ether, pentaerythritol trivinyl ether, pentaerythritol tetravinyl ether, sorbitol tetravinyl ether, sorbitol pentavinyl ether, ethylene glycol diethylene vinyl ether, triethylene glycol diethylene vinyl ether, ethylene glycol dipropylene vinyl ether, triethylene glycol diethylene vinyl ether, trimethylolpropane triethylene vinyl ether, trimethylolpropane diethylene vinyl ether, pentaerythritol diethylene vinyl ether, pentaerythritol triethylene vinyl ether, pentaerythritol tetraethylene vinyl ether, 1,1,1-tris[4-(2-vinyloxyethoxy)phenyl]ethane, bisphenol A divinyloxyethyl ether, etc.

These vinyl ether compounds can be produced, for example, according to the method described in Stephen. C. Lapin, Polymers Paint Colour Journal, 179 (4237), 321 (1988), concretely through reaction of a polyalcohol or a polyphenol with acetylene, or through reaction of a polyalcohol or a polyphenol with a halogenoalkyl vinyl ether. One or more of these compounds may be used either singly or as combined.

Examples of the styrene derivatives used with monofunctional monomer of the invention include styrene, p-methylstyrene, p-methoxystyrene, β-methylstyrene, p-methyl-β-methylstyrene, α-methylstyrene, p-methoxy-β-methylstyrene and p-hydroxystyrene. Examples of vinylnaphthalene derivatives include 1-vinylnaphthalene, α-methyl-1-vinylnaphthalene, β-methyl-1-vinylnaphthalene, 4-methyl-1-vinylnaphthalene and 4-methoxyl-1-vinylnaphthalene.

On the other hand, it is desirable that a styrene derivative is not used as the polymerizable monomer in the composition of the invention. This is because, when a styrene derivative is incorporated in the composition of the invention, then the photocurability of the composition may worsen enormously and the formed resist pattern may be thereby tacky.

For the purpose of enhancing the mold releasability and the coatability, a fluorine atom-having compound may be combined with the polymerizable monomer in the composition of the invention. The compound includes, for example, trifluoroethyl (meth)acrylate, pentafluoroethyl (meth)acrylate, (perfluorobutyl)ethyl (meth)acrylate, per fluorobutyl-hydroxypropyl (meth)acrylate, (perfluorohexyl)ethyl (meth)acrylate, octafluoropentyl (meth)acrylate, perfluorooctylethyl (meth)acrylate, tetrafluoropropyl (meth)acrylate, etc.

Other polymerizable monomers such as propenyl ether and butenyl ether may be incorporated in the composition of the invention. For example, preferred are 1-dodecyl-1-propenyl ether, 1-dodecyl-1-butenyl ether, 1-butenoxymethyl-2-norbornene, 1,4-di(1-butenoxy)butane, 1,10-di(butenoxy)decane, 1,4-di(1-butenoxymethyl)cyclohexane, diethylene glycol di(1-butenyl)ether, 1,2,3-tri(1-butenoxy)propane, propenyl ether propylene carbonate, etc.

The compound of the compound except the (meth)acrylate compound of formula (1) in the composition of the invention may be generally from 10 to 80% by mass of the composition, preferably from 30 to 75% by mass.

### (Polymerization Initiator)

The composition of the invention generally contains a photopolymerization initiator. The content of the photopolymerization initiator in the composition of the invention may be, for example, from 0.1 to 15% by mass, preferably from 0.2 to 12% by mass, more preferably from 0.3 to 10% by mass. In case where two or more different types of photopolymerization initiators are in the composition, their total amount may fall within the above range.

When the proportion of the photopolymerization initiator is at least 0.1% by mass, then it is favorable since the composition tends to better in point of the sensitivity (rapid curability), the resolution, the line edge roughness reduction and the coating film strength. On the other hand, when the proportion of the photopolymerization initiator is at most 15% by mass, then it is also favorable since the composition tends to better in point of the light transmittance, the discoloration resistance and the handlability.

The photopolymerization initiator for use in the invention includes those active to the wavelength of the light source to be used, for which, for example, usable are those capable of generating suitable active species. One or more different types of photopolymerization initiators may be used either singly or as combined.

As the radical photopolymerization initiator for use in the invention, for example, commercial products may be used. Their examples are Irgacure® 2959 (1-[4-(2-hydroxyethoxy)phenyl]-2-hydroxy-2-methyl-1-propan-1-one), Irgacure® 184 (1-hydroxycyclohexyl phenyl ketone), Irgacure® 500 (1-hydroxycyclohexyl phenyl ketone, benzophenone), Irgacure® 651 (2,2-dimethoxy-1,2-diphenylethan-1-one), Irgacure® 369 (2-benzyl-2-dimethylamino-1-(4-morpholinophenyl)butanone-1), Irgacure® 907 (2-methyl-1[4-methylthiophenyl]-2-morpholinopropan-1-one), Irgacure® 819 (bis(2,4,6-trimethylbenzoyl)-phenylphosphine oxide), Irgacure® 1800 (bis(2,6-dimethoxybenzoyl)-2,4,4-trimethylpentylphosphine oxide, 1-hydroxycyclohexyl phenyl ketone), Irgacure® 1800 (bis(2,6-dimethoxybenzoyl)-2,4,4-trimethylpentylphosphine oxide, 2-hydroxy-2-methyl-1-phenyl-1-propan-1-one), Irgacure® OXE01 (1,2-octanedione, 1-[4-(phenylthio)phenyl]-2-(O-benzoyloxime)), Darocur® 1173 (2-hydroxy-2-methyl-1-phenyl-1-propan-1-one), Darocur® 1116, 1398, 1174 and 1020, CGI242 (ethanone, 1-[9-ethyl-6-(2-methylbenzoyl)-9H-carbazol-3-yl]-1-(O-acetyloxime)), which are all available from Ciba; Lucirin TPO (2,4,6-trimethylbenzoyldiphenylphosphine oxide), Lucirin TPO-L (2,4,6-trimethylbenzoylphenylethoxyphosphine oxide) which are both available from BASF; Esacure 1001M (1-[4-benzoylphenylsulfanyl]phenyl)-2-methyl-2-(4-methylphenylsulfonyl)propan-1-one available from Nihon SiberHegner; Adeka Optomer® N-1414 (carbazole/phenone compound), Adeka Optomer® N-1717 (acridine compound), Adeka Optomer® N-1606 (triazine compound), all available from Asahi Denka; Sanwa Chemical's TFE-triazine (2-[2-(furan-2-yl)vinyl]-4,6-bis(trichloromethyl)-1,3,5-tri azine), Sanwa Chemical's TME-triazine (2-[2-(5-methylfuran-2-yl)vinyl]-4,6-bis(trichloromethyl)-1,3,5-triazine), Sanwa Chemical's MP-triazine (2-(4-methoxyphenyl)-4,6-bis(trichloromethyl)-1,3,5-triazine); Midori Chemical's TAZ-113 (2-[2-(3,4-dimethoxyphenyl)ethenyl]-4,6-bis(trichloromethyl)-1,3,5-triazine), Midori Chemical's TAZ-108 (2-(3,4-dimethoxyphenyl)-4,6-bis(trichloromethyl)-1,3,5-tri azine; as well as benzophenone, 4,4′-bisdiethylaminobenzophenone, methyl-2-benzophenone, 4-benzoyl-4′-methyldiphenyl sulfide, 4-phenylbenzophenone, ethyl Michler's ketone, 2-chlorothioxanthone, 2-methylthioxanthone, 2-isopropylthioxanthone, 4-isopropylthioxanthone, 2,4-diethylthioxanthone, 1-chloro-4-propoxythioxanthone, 2-methylthioxanthone, thioxanthone ammonium salt, benzoin, 4,4′-dimethoxybenzoin, benzoin methyl ether, benzoin ethyl ether, benzoin isopropyl ether, benzoin isobutyl ether, benzyl dimethyl ketal, 1,1,1-trichloroacetophenone, diethoxyacetophenone, dibenzosuberone, methyl o-benzoylbenzoate, 2-benzoylnaphthalene, 4-benzoylbiphenyl, 4-benzoyldiphenyl ether, 1,4-benzoylbenzene, benzil, 10-butyl-2-chloroacridone, [4-(methylphenylthio)phenyl]phenylmethane), 2-ethylanthraquinone, 2,2-bis(2-chlorophenyl)-4,5,4′,5′-tetrakis(3,4,5-trimethoxy phenyl)-1,2′-biimidazole, 2,2-bis(o-chlorophenyl)-4,5,4′,5′-tetraphenyl-1,2′-biimidazole, tris(4-dimethylaminophenyl)methane, ethyl 4-(dimethylamino)benzoate, 2-(dimethylamino)ethyl benzoate, butoxyethyl 4-(dimethylamino)benzoate, etc.

The light for polymerization initiation in the invention includes not only light having a wavelength falling within a range of UV light, near-UV light, far-UV light, visible light, IR light, etc., but also electromagnetic waves and radiations. The radiations include, for example, microwaves, electron beams, EUV, and X rays. In addition, laser light such as 248 nm excimer laser, 193 nm excimer laser, 172 nm excimer laser, etc. The light may be a monochromatic light (light having a single wavelength) having passed through an optical filter, or may be a composite light (mixed light) of multiple rays each having a different wavelength. The photoexposure may be multi-stage photoexposure, and for the purpose of enhancing the film strength and the etching resistance, the patterned film may be further photoexposed on the entire surface thereof.

In the invention, the type of the photopolymerization initiator to be used shall be suitably selected depending on the wavelength of the light source to be used, but is preferably one not generating gas during mold pressing and photoexposure. Gas generation, if any, may cause mold contamination, therefore giving problems in that the mold must be washed frequently and the curable nanoimprint composition of the invention may be deformed in the mold and the transferred pattern accuracy may be thereby worsened. The photopolymerization initiator for use in the invention is preferably one not generating gas, since the initiator of the type is free from the trouble of mold contamination therefore not increasing mold washing frequency; and accordingly, the initiator of the type is favorable from the viewpoint that the curable nanoimprint composition of the invention hardly deforms in a mold and therefore does not worsen the transfer patterning accuracy.

### (Antioxidant)

The composition of the invention may contain a known antioxidant. The content of the antioxidant to be in the composition of the invention is, for example, from 0.01 to 10% by mass of the total amount of composition, preferably from 0.2 to 5% by mass. When two or more different types of antioxidants are in the composition, the total amount thereof falls within the above range.

The antioxidant is for preventing fading by heat or photoirradiation, and for preventing fading by various oxidizing gases such as ozone, active hydrogen, NOx, SOx (x is an integer), etc. Especially in the invention, the antioxidant added to the composition brings about the advantage that the cured film is prevented from being discolored and the film thickness is prevented from being reduced through decomposition. The antioxidant includes hydrazides, hindered amine-type antioxidants, nitrogen-containing heterocyclic mercapto compounds, thioether-type antioxidants, hindered phenol-type antioxidants, ascorbic acids, zinc sulfate, thiocyanates, thiourea derivatives, saccharides, nitrites, sulfites, thiosulfates, hydroxylamine derivatives, etc. Of those, preferred are hindered phenol-type antioxidants and thioether-type antioxidants from the viewpoint of their effect of preventing cured film discoloration and preventing film thickness reduction.

Commercial products of the antioxidant usable herein include Irganox 1010, 1035, 1076, 1222 (all by Ciba-Geigy); Antigene P, 3C, FR, Sumilizer S, Sumilizer GA80 (by Sumitomo Chemical); Adekastab A070, A080, A0503 (by Adeka), etc. These may be used either singly or as combined.

### (Surfactant)

The composition the invention may contain a surfactant. The content of the surfactant that may be in the composition may be, for example, from 0.001 to 5% by mass of the total amount of the composition, preferably from 0.002 to 4% by mass, more preferably from 0.005 to 3% by mass. In case where two or more different types of surfactants are in the composition, the total amount thereof falls within the above range. When the surfactant content in the composition is less than 0.001% by mass, then the coating uniformity of the composition may be poor; but on the other hand, when the content is more than 5% by mass, then it may worsen the mold transferability and is therefore unfavorable.

As the surfactant, preferably, the composition contains at least one of a fluorine-containing surfactant, a silicone-type surfactant and a fluorine-containing silicone-type surfactant, more preferably contains both of a fluorine-containing surfactant and a silicone-type surfactant, or contains a fluorine-containing silicone-type surfactant, most preferably contains a fluorine-containing silicone-type surfactant.

The fluorine-containing silicone-type surfactant as referred to herein means a surfactant satisfying both the requirement of a fluorine-containing surfactant and that of a silicone-type surfactant.

Using the surfactant of the type may solve the problem of coating failures such as striation and flaky pattern formation (drying unevenness of resist film) that may occur when the composition of the invention is applied onto substrates on which various films are formed, for example, onto silicon wafers in semiconductor production, or onto glass square substrates, chromium films, molybdenum films, molybdenum alloy films, tantalum films, tantalum alloy films, silicon nitride films, amorphous silicon films, tin oxide-doped indium oxide (ITO) films or tin oxide films in production of liquid-crystal devices. In addition, the surfactant is effective for enhancing the flowability of the composition of the invention in the cavity of the concave part of mold, for enhancing the mold-resist releasability, for enhancing the resist adhesiveness to substrates, and for lowering the viscosity of the composition.

Examples of the nonionic fluorine-containing surfactant usable in the invention include Fluorad FC-430, FC-431 (Sumitomo 3M's trade names); Surflon S-382 (Asahi Glass's trade name); EFTOP EF-122A, 122B, 122C, EF-121, EF-126, EF-127, MF-100 (Tochem Products' trade names); PF-636, PF-6320, PF-656, PF-6520 (OMNOVA Solution's trade names); Futagent FT250, FT251, DFX18 (Neos' trade names); Unidyne DS-401, DS-403, DS-451 (Daikin's trade names); Megafac 171, 172, 173, 178K, 178A, F780F (Dai-Nippon Ink's trade names). Examples of the nonionic silicone-type surfactant include SI-10 series, Pionin D6315 (Takemoto Yushi's trade name), Megafac Paintad 31 (Dai-Nippon Ink's trade name), KP-341 (Shin-Etsu Chemical's trade name).

Examples of the fluorine-containing silicone-type surfactant for use in the invention include X-70-090, X-70-091, X-70-092, X-70-093 (Shin-Etsu Chemical's trade names); Megafac R-08, XRB-4 (Dai-Nippon Ink's trade names).

The surfactant for use in the composition of the invention is preferably a nonionic surfactant from the viewpoint of the voltage retentiveness.

### Other Ingredients:

In addition to the above-mentioned ingredients, the composition of the invention may contain, if desired, release agent, silane coupling agent, UV absorbent, light stabilizer, antiaging agent, plasticizer, adhesion promoter, thermal polymerization initiator, colorant, elastomer particles, photoacid enhancer, photobase generator, basic compound, flowability promoter, defoaming agent, dispersant, etc.

For the purpose of further enhancing the releasability thereof, the composition of the invention may contain a release agent. Concretely, the release agent is added to the composition for the purpose of smoothly releasing the mold pressed to the layer of the composition of the invention not causing surface roughening of the resin layer and not deforming the pattern formed on the layer. The release agent may be any known release agent, including, for example, silicone release agents, solid waxes such as polyethylene wax, amide wax, Teflon® powder, etc., as well as fluorine-containing compounds, phosphate compounds, etc. The release agent may be previously applied to the surface of a mold.

A silicone release agent exhibits especially good mold releasability when combined with the above-mentioned photocurable resin for use in the invention, hardly causing pattern deformation in mold releasing. The silicone release agent has a basic structure of an organopolysiloxane structure, including, for example, native or denatured silicone oil, trimethylsiloxysilicic acid-containing polysiloxane, silicone-type acrylic resin, etc. Ordinary silicone-type leveling agent generally used in hard coat compositions is also usable herein.

Denatured silicone oil is one produced by denaturing the side branch and/or the terminal of polysiloxane, and is grouped into reactive silicone oil and non-reactive silicone oil. The reactive silicone oil includes amino-modified, epoxy-modified, carboxyl-modified, carbinol-modified, methacryl-modified, mercapto-modified, phenol-modified, semiterminal-modified, heterofunctional group-modified ones. The non-reactive silicone oil includes polyether-modified, methylstyryl-modified, alkyl-modified, higher fatty ester-modified, hydrophilic specific-modified, higher alkoxy-modified, higher fatty acid-modified, fluorine-modified ones.

One polysiloxane molecule may have two or more of the above-mentioned denaturation modes.

Preferably, the denatured silicone oil has suitable compatibility with the constitutive ingredients of the composition of the invention. In particular, in case where a reactive silicone oil that is reactive with the optional constitutive ingredients in the composition of the invention is used in the composition, it may be fixed by chemical bonding in the cured film of the composition, and therefore it may be free from a problem of worsening the adhesiveness of the cured film and from other problems of contamination and degradation of the cured film. In particular, the reactive silicone oil is effective for enhancing the adhesiveness to the vapor-deposition film in an evaporation step. A silicone modified with a photocurable functional group, such as a (meth)acryloyl-modified silicone, a vinyl-modified silicone or the like, may crosslink with the constitutive ingredients of the composition of the invention, and therefore it may enhance the properties of the cured film.

A trimethylsiloxysilicic acid-containing polysiloxane may readily bleed out on the surface of the composition and therefore brings about excellent releasability. In addition, even though it bleeds out on the surface, the polysiloxane of the type still keeps good adhesiveness and is excellent in adhesiveness to a metal-deposition layer and an overcoat layer, and therefore it is favorable for use herein.

One or more different types of the above-mentioned release agents may be used herein either singly or as combined.

In case where the release agent is added to the composition of the invention, its content is preferably from 0.001 to 10% by mass of the total amount of the composition, more preferably from 0.01 to 5% by mass. When the proportion of the release agent is less than the above range, then the mold releasability of the layer of the curable composition for photonanoimprints of the invention may be poor; and when the proportion is more than the range, then it may unfavorably cause a problem of surface roughening of the coating layer as the composition may be repelled in coating, or may cause other problems of worsening the adhesiveness of the composition to substrates themselves or to adjacent layers such as deposit layers in the products or breaking the coating film in transferring (as the film strength is too weak).

When the proportion of the release agent is at least 0.01% by mass, then the mold releasability of the layer of the curable composition for photonanoimprints of the invention may be further better. On the other hand, when the proportion is at most 10% by mass, then it is favorable since it does not cause a problem of surface roughening of the coating layer as the composition may be prevented from being repelled in coating, or does not cause other problems of worsening the adhesiveness of the composition to substrates themselves or to adjacent layers such as deposit layers in the products or breaking the coating film in transferring (as the film strength is too weak).

The composition of the invention may contain an organic metal coupling agent added thereto, for the purpose of enhancing the heat resistance and the strength of the micropatterned surface structure of the layer of the composition and for enhancing the adhesiveness of the layer to metal deposition layers. The organic metal coupling agent is effective, as having the effect of promoting the thermosetting reaction of the composition. As the organic metal coupling agent, herein usable are various coupling agents such as a silane coupling agent, a titanium coupling agent, a zirconium coupling agent, an aluminium coupling agent, a tin coupling agent, etc.

The silane coupling agent for use in the composition of the invention includes, for example, vinylsilanes such as vinyltrichlorosilane, vinyltris(β-methoxyethoxy)silane, vinyltriethoxysilane, vinyltrimethoxysilane, etc.; epoxysilanes such as β-(3,4-epoxycyclohexyl)ethyltrimethoxysilane, γ-glycidoxypropyltrimethoxysilane, γ-glycidoxypropylmethyldiethoxysilane, etc.; aminosilanes such as N-β-(aminoethyl)-γ-aminopropyltrimethoxysilane, N-β-(aminoethyl)-γ-aminopropylmethyldimethoxysilane, γ-aminopropyltrimethoxysilane, N-phenyl-γ-aminopropyltrimethoxysilane, etc.; and other silane coupling agents such as γ-mercaptopropyltrimethoxysilane, 7-chloropropylmethyldimethoxysilane, γ-chloropropylmethyldiethoxysilane, etc.

The titanium coupling agent includes, for example, isopropyltriisostearoyl titanate, isopropyltridecylbenzenesulfonyl titanate, isopropyltris(dioctylpyrophosphate) titanate, tetraisopropylbis(dioctylphosphite) titanate, tetraoctylbis(ditridecylphosphite) titanate, tetra(2,2-diallyloxymethyl)bis(ditridecyl)phosphite titanate, bis(dioctylpyrophosphate)oxyacetate titanate, bis(dioctylpyrophosphate)ethylene titanate, isopropyltrioctanoyl titanate, isopropyldimethacrylisostearoyl titanate, isopropylisostearoyldiacryl titanate, isopropyltri(dioctylphosphate) titanate, isopropyltricumyl titanate, isopropyltri(N-aminoethyl-aminoethyl) titanate, dicumylphenyloxyacetate titanate, diisostearoylethylene titanate, etc.

The zirconium coupling agent includes, for example, tetra-n-propoxy zirconium, tetra-butoxy zirconium, zirconium tetraacetylacetate, zirconium dibutoxybis(acetylacetonate), zirconium tributoxyethyl acetacetate, zirconium butoxyacetylacetonate bis(ethylacetacetate), etc.

The aluminium coupling agent includes, for example, aluminium isopropylate, mono-sec-butoxyaluminium diisopropylate, aluminium sec-butyrate, aluminium ethylate, ethylacetacetate aluminium diisopropylate, aluminium tris(ethylacetacetate), alkylacetacetate aluminium diisopropylate, aluminium monoacetylacetonate bis(ethylacetacetate), aluminium tris(acetylacetate), etc.

The above-mentioned organic metal coupling agent may be in the curable composition for photonanoimprint lithography in an amount falling within a range of from 0.001 to 10% by mass of the total solid content of the composition. When the proportion of the organic metal coupling agent is at least 0.001% by mass, then it may be more effective for enhancing the heat resistance, the strength and the adhesiveness to deposition layers of the cured film. On the other hand, when the proportion of the organic metal coupling agent is at most 10% by mass, then it is favorable since the composition may be stabilized and may have good film formability.

Commercial products of UV absorbent usable herein include Tinuvin P, 234, 320, 326, 327, 328, 213 (all by Ciba-Geigy), Sumisorb 110, 130, 140, 220, 250, 300, 320, 340, 350, 400 (all by Sumitomo Chemical Industry), etc. Preferably, the UV absorbent is in the curable composition for photonanoimprint lithography in an amount falling within a range of from 0.01 to 10% by mass of the total amount of the composition.

Commercial products of light stabilizer usable herein include Tinuvin 292, 144, 622 LD (all by Ciba-Geigy), Sanol LS-770, 765, 292, 2626, 1114, 744 (all by Sankyo Chemical Industry), etc. Preferably, the light stabilizer is in the composition in an amount falling within a range of from 0.01 to 10% by mass of the total amount of the composition.

Commercial products of antiaging agent usable herein include Antigene W, S, P, 3C, 6C, RD-G, FR, AW (all by Sumitomo Chemical Industry), etc. Preferably, the antiaging agent is in the composition in an amount falling within a range of from 0.01 to 10% by mass of the total amount of the composition.

A plasticizer may be added to the composition of the invention for the purpose of controlling the adhesiveness of the composition to substrate, and the flexibility and the hardness of the formed film, etc. Preferred examples of the plasticizer include, for example, dioctyl phthalate, didodecyl phthalate, triethylene glycol dicaprylate, dimethylglycol phthalate, tricresyl phosphate, dioctyl adipate, dibutyl sebacate, triacetylglycerine, dimethyl adipate, diethyl adipate, di(n-butyl)adipate, dimethyl suberate, diethyl suberate, di(n-butyl)suberate, etc. The plasticizer may be in the composition in an amount of at most 30% by mass of the composition. Preferably, the amount is at most 20% by mass, more preferably at most 10% by mass. In order that the plasticizer could exhibit the desired effect thereof, its amount is preferably at least 0.1% by mass.

An adhesiveness promoter may be added to the composition of the invention for the purpose of controlling the adhesiveness of the composition to substrate. The adhesiveness promoter includes benzimidazoles, polybenzimidazoles, lower hydroxyalkyl-substituted pyridine derivatives, nitrogen-containing heterocyclic compounds, urea or thiourea, organic phosphorous compounds, 8-hydroxyquinoline, 4-hydroxypteridine, 1,10-phenanthroline, 2,2′-bipyridine derivatives, benzotriazoles, organic phosphorus compounds, phenylenediamine compounds, 2-amino-1-phenylethanol, N-phenylethanolamine, N-ethyldiethanolamine, N-ethyldiethanolamine, N-ethylethanolamine and its derivatives, benzothiazole derivatives, etc. The adhesiveness promoter may be in the composition preferably in an amount of at most 20% by mass of the composition, more preferably at most 10% by mass, even more preferably at most 5% by mass. For attaining the effect of the adhesiveness promoter, the amount thereof is preferably at least 0.1% by mass.

In case where the composition of the invention is cured, a thermal polymerization initiator may be added thereto, if desired. Preferred examples of the thermal polymerization initiator include, for example, peroxides and azo compounds. Specific examples of the compounds are benzoyl peroxide, tert-butyl peroxybenzoate, azobisisobutyronitrile, etc.

A photobase generator may be added, if desired, to the composition of the invention for the purpose of controlling the patterning profile and the sensitivity of the composition. Preferred examples of the photobase generator include 2-nitrobenzylcyclohexyl carbamate, triphenylmethanol, O-carbamoylhydroxylamide, O-carbamoyl oxime, [[(2,6-dinitrobenzyl)oxy]carbonyl]cyclohexylamine, bis[[(2-nitrobenzyl)oxy]carbonyl]hexane-1,6-diamine, 4-(methylthiobenzoyl)-1-methyl-1-morpholinoethane, (4-morpholinobenzoyl)-1-benzyl-1-dimethylaminopropane, N-(2-nitrobenzyloxycarbonyl)pyrrolidine, hexaamine-cobalt(III)tris(triphenylmethyl borate), 2-benzyl-2-dimethylamino-1-(4-morpholinophenyl)-butanone, 2,6-dimethyl-3,5-diacetyl-4-(2′-nitrophenyl)-1,4-dihydropyridine, 2,6-dimethyl-3,5-diacetyl-4-(2′,4′-dinitrophenyl)-1,4-dihydropyridine, etc.

A colorant may be optionally added to the composition of the invention for the purpose of enhancing the visibility of the coating film. As the colorant, herein usable are pigments and dyes that are used in UV inkjet compositions, color filter compositions, CCD image sensor compositions and the like, not detracting from the object of the invention. The pigments usable in the invention may be various known inorganic pigments and organic pigments. The inorganic pigments include metal compounds such as metal oxides, metal complexes and others, concretely iron, cobalt, aluminium, cadmium, lead, copper, titanium, magnesium, chromium, zinc, antimony and the like metal oxides, metal composite oxides, etc. Examples of the organic pigments include C.I. Pigment Yellow 11, 24, 31, 53, 83, 99, 108, 109, 110, 138, 139, 151, 154, 167, C.I. Pigment Orange 36, 38, 43, C.I. Pigment Red 105, 122, 149, 150, 155, 171, 175, 176, 177, 209, C.I. Pigment Violet 19, 23, 32, 39, C.I. Pigment Blue 1, 2, 15, 16, 22, 60, 66, C.I. Pigment Green 7, 36, 37, C.I. Pigment Brown 25, 28, C.I. Pigment Black 1, 7 and carbon black. Preferably, the colorant may be in the composition in an amount of from 0.001 to 2% by mass of the total amount of the composition.

As still another optional ingredient, elastomer particles may be added to the composition of the invention for the purpose of enhancing the mechanical strength and the flexibility of the cured film.

The elastomer particles capable of being optionally added to the composition of the invention preferably have a mean particle size of from 10 nm to 700 nm, more preferably from 30 to 300 nm. For example, the elastomer particles are of polybutadiene, polyisoprene, butadiene/acrylonitrile copolymer, styrene/butadiene copolymer, styrene/isoprene copolymer, ethylene/propylene copolymer, ethylene/α-olefin copolymer, ethylene/α-olefin/polyene copolymer, acrylic rubber, butadiene/(meth)acrylate copolymer, styrene/butadiene block copolymer, styrene/isoprene block copolymer, etc. Also usable are core/shell particles prepared by coating these elastomer particles with methyl methacrylate polymer, methyl acrylate/glycidyl methacrylate copolymer or the like. The elastomer particles may have a crosslinked structure.

Commercial products of elastomer particles usable herein are, for example, Resinous Bond RKB (by Resinous Chemical), Techno MBS-61, MBS-69 (by Techno Polymer), etc.

One or more different types of these elastomer particles may be in the composition of the invention either singly or as combined. The content of the elastomer particles in the composition may be preferably from 1 to 35% by mass, more preferably from 2 to 30% by mass, even more preferably from 3 to 20% by mass.

A basic compound may be optionally added to the composition of the invention for the purpose of preventing the composition from shrinking in curing and for enhancing the thermal stability of the composition. The basic compound includes amines, nitrogen-containing heterocyclic compounds such as quinoline, quinolidine, etc., and basic alkali metal compounds, basic alkaline earth metal compounds, etc. Of those, preferred are amines from the viewpoint of the compatibility thereof with photopolymerizable monomers; and for example, they include octylamine, naphthylamine, xylenediamine, dibenzylamine, diphenylamine, dibutylamine, dioctylamine, dimethylaniline, quinuclidine, tributylamine, trioctylamine, tetramethylethylenediamine, tetramethyl-1,6-hexamethylenediamine, hexamethylenetetramine, triethanolamine, etc.

A chain transfer agent may be added to the composition of the invention for enhancing the photocurability of the composition. Concretely, the agent includes 4-bis(3-mercaptobutyryloxy)butane, 1,3,5-tris(3-mercaptobutyloxyethyl)-1,3,5-triazine-2,4,6-(1H,3H,5H)-trione, pentaerythritol tetrakis(3-mercaptobutyrate).

### (Organic Solvent)

Preferably, the content of the organic solvent in the composition of the invention is at most 3% by mass of the total composition. Specifically, since the composition of the invention preferably contains a specific monofunctional and/or bifunctional monomer as a reactive diluent, the composition does not always require an organic solvent for dissolving the constitutive ingredients therein. When the composition does not contain an organic solvent, then it does not require a baking step for evaporating the solvent, and the absence of a solvent in the composition is advantageous for simplifying the process for the composition. Accordingly, the content of the organic solvent in the composition of the invention is preferably at most 3% by mass, more preferably at most 2% by mass, and even more preferably, the composition contains no solvent. To that effect, the composition of the invention does not always contain an organic solvent; however, in case where the composition comprises compounds insoluble in the reactive diluent therein or in case where the viscosity of the composition is delicately controlled, an organic solvent may be optionally added to the composition. The organic solvent favorable for use in the composition of the invention may be any one generally used in ordinary curable compositions for nanoimprints or in photoresists. Not specifically defined, therefore, the organic solvent may be any one capable of uniformly dissolving and dispersing the constitutive ingredients of the composition of the invention but not reacting with those ingredients.

The organic solvent includes, for example, alcohols such as methanol, ethanol, etc.; ethers such as tetrahydrofuran, etc.; glycol ethers such as ethylene glycol monomethyl ether, ethylene glycol dimethyl ether, ethylene glycol methylethyl ether, ethylene glycol monoethyl ether, etc.; ethylene glycol alkyl ether acetates such as methyl cellosolve acetate, ethyl cellosolve acetate, etc.; diethylene glycols such as diethylene glycol monomethyl ether, diethylene glycol diethyl ether, diethylene glycol dimethyl ether, diethylene glycol ethylmethyl ether, diethylene glycol monomethyl ether, diethylene glycolmonobutyl ether, etc.; propylene glycol alkyl ether acetates such as propylene glycol methyl ether acetate, propylene glycol ethyl ether acetate, etc.; aromatic hydrocarbons such as toluene, xylene, etc.; ketones such as acetone, methyl ethyl ketone, methyl isobutyl ketone, cyclohexanone, 4-hydroxy-4-methyl-2-pentanone, 2-heptanone, etc.; esters such as ethyl 2-hydroxypropionate, methyl 2-hydroxy-2-methylpropionate, ethyl 2-hydroxy-2-methylpropionate, ethyl hydroxyacetate, ethyl hydroxyacetate, methyl 2-hydroxy-2-methylbutanoate, methyl 3-methoxypropionate, ethyl 3-methoxypropionate, methyl 3-ethoxypropionate, ethyl 3-ethoxypropionate, ethyl acetate, butyl acetate, methyl lactate, ethyl lactate, other lactates, etc.

In addition, a high-boiling-point solvent may also be added to the composition; and the solvent includes N-methylformamide, N,N-dimethylformamide, N-methylformanilide, N-methylacetamide, N,N-dimethylacetamide, N-methylpyrrolidone, dimethyl sulfoxide, benzyl ethyl ether, dihexyl ether, acetonylacetone, isophorone, caproic acid, caprylic acid, 1-octanol, 1-nonanol, benzyl alcohol, benzyl acetate, ethyl benzoate, diethyl oxalate, diethyl maleate, γ-butyrolactone, ethylene carbonate, propylene carbonate, phenyl cellosolve acetate, etc. One or more of these solvents may be in the composition of the invention either singly or as combined.

Of those, especially preferred are methoxypropylene glycol acetate, ethyl 2-hydroxypropionate, methyl 3-methoxypropionate, ethyl 3-ethoxypropionate, ethyl lactate, cyclohexanone, methyl isobutyl ketone, 2-heptanone, etc.

### (Viscosity)

The viscosity of the composition is described. Unless otherwise specifically indicated, the viscosity in the invention is at 25° C. The viscosity at 25° C. of the composition of the invention from which the solvent is removed is generally at most 18 mPa·s, preferably at most 12 mPa·s. Not specifically defined, the lowermost limit is preferably at least 3 mPa·s. When the viscosity of the composition of the invention is at least 3 mPa·s, then the composition may be free from a problem of coating failure on substrates and the mechanical strength of the coating film may be high. Concretely, when the viscosity is at least 3 mPa·s, then it is favorable since the composition is free from a problem of coating unevenness and a problem of flowing out on substrate in application thereto. On the other hand, when the viscosity of the composition is at most 18 mPa·s, then it is favorable because of the following reasons. When a mold having a micropattern is airtightly attached to the composition, the composition may well flow into the cavity of the concave part of the mold not taking bubbles therein, and therefore, the composition is free from a problem of bubble defects and, after cured, the composition leaves few residues in the concave part of the mold.

### (Surface Tension)

Preferably, the composition of the invention has a surface tension falling within a range of from 18 to 30 mN/m, more preferably from 20 to 28 mN/m. When the surface tension of the composition falls within the range, then the cured film may have good surface smoothness.

### (Water Content)

The water content of the composition of the invention in its preparation is preferably at most 2.0% by mass, more preferably at most 1.5% by mass, even more preferably at most 1.0% by mass. When the water content of the composition in its preparation is at most 2.0% by mass, then the storage stability of the composition may be bettered.

### (Preparation)

The composition of the invention may be prepared by mixing the above-mentioned ingredients followed by filtering the resulting mixture through a filter having a pore size of from 0.05 μm to 5.0 μm to give a solution. Mixing and dissolving the ingredients to give the curable composition for nanoimprints may be attained generally at a temperature falling within a range of from 0° C. to 100° C. The filtration may be attained in multiple stages, or may be repeated multiple times. The filtrate may be re-filtered. Not specifically defined, the material for use for filtration may be any of polyethylene resin, polypropylene resin, fluororesin, nylon resin, etc.

### [Cured Film]

Next described is the cured film of the invention (especially having a micropattern) formed of the composition of the invention. In the invention, the composition of the invention may be applied onto a substrate and cured to form thereon a cured film of the invention.

The composition of the invention may be applied onto a substrate or a support, then the layer of the composition may be exposed to light, cured and optionally baked, thereby forming a permanent film such as an overcoat layer, an insulation film or the like.

In permanent films (resists for constitutive components) for use in liquid-crystal displays (LCD), the resist is preferably prevented from being contaminated as much as possible with metallic or organic ionic impurities in order that the resist does not interfere with the performance of the displays. Accordingly, the concentration of the impurities is preferably at most 1000 ppm, more preferably at most 100 ppm.

After its production, the composition for permanent films (resists for constitutive components) for use in liquid-crystal displays (LCD) may be bottled in a container such as a gallon bottle or a coated bottle, and may be transported or stored. In this case, the container may be purged with an inert gas such as nitrogen, argon or the like for preventing the composition therein from being degraded. The composition may be transported or stored at ordinary temperature, but for preventing the permanent film to be formed of the composition from being degraded, it is preferably transported or stored at a controlled temperature of from −20° C. to 0° C. Needless-to-say, the composition must be shielded from light to such a level on which its reaction does not go on.

### [Components for Liquid-Crystal Display Devices]

The cured product of the invention is favorably sued for semiconductor integrated circuits, recording materials, components for liquid-crystal display devices, etc. In particular, it is favorable for components for liquid-crystal display devices, and is especially preferred as an etching resist for sue in flat panel displays, etc.

### [Production Method for Cured Film]

A production method for a cured film using the composition of the invention is described below.

The composition of the invention is preferably cured with light, or with light and heat. Concretely, at least a composition of the invention is applied onto a substrate, then the solvent is dried to form a layer of the composition of the invention to give a pattern receptor, a mold is pressed against the pattern-forming surface of the pattern receptor, and processed for mold pattern transferring thereonto, and the resulting micropattern is cured through photoirradiation and heating. In general, photoirradiation and heating are attained multiple times. The nanoimprint lithography in the production method for the cured film of the invention may attain lamination and multiple patterning, and may be combined with ordinary thermal imprinting.

To form the cured film, the composition of the invention may be applied onto a substrate in any well-known method, for example, a dip coating method, an air knife coating method, a curtain coating method, a wire bar coating method, a gravure coating method, an extrusion coating method, a spin coating method, a slit scanning method or the like. The thickness of the layer formed of the composition of the invention may vary depending on the use thereof, and may be from 0.05 μm to 30 μm. The composition of the invention may be used in multi-stage coating.

Not specifically defined, the substrate to which the composition of the invention is applied may be any of quartz, glass, optical film, ceramic material, vapor deposition film, magnetic film, reflective film, metal substrate of Ni, Cu, Cr, Fe or the like, paper, SOG, polymer substrate such as polyester film, polycarbonate film, polyimide film or the like, TFT array substrate, PDP electrode plate, glass or transparent plastic substrate, electroconductive substrate of ITO, metal or the like, insulating substrate, semiconductor substrate such as silicon, silicon nitride, polysilicon, silicon oxide, amorphous silicon or the like. The shape of the substrate may be tabular or roll.

Light to cure the composition of the invention is not specifically defined. For example, it includes light and irradiations with a wavelength falling within a range of high-energy ionizing radiation, near-ultraviolet, far-ultraviolet, visible, infrared, etc. The high-energy ionizing radiation source includes, for example, accelerators such as Cockcroft accelerator, Handegraf accelerator, linear accelerator, betatoron, cyclotron, etc. The electron beams accelerated by such an accelerator are used most conveniently and most economically; however, any other radioisotopes and other radiations are also usable, which are from nuclear reactors, such as γ rays, X rays, α rays, neutron beams, proton beams, etc. The UV sources include, for example, UV fluorescent lamp, low-pressure mercury lamp, high-pressure mercury lamp, ultra-high-pressure mercury lamp, xenon lamp, carbon arc lamp, solar lamp, etc. The radiations include microwaves, EUV, etc. In addition, laser rays for use in microprocessing of semiconductors, such as LED, semiconductor laser ray, 248 nm KrF excimer laser ray, 193 nm ArF excimer laser ray and others, are also favorably used in the invention. These lights may be monochromatic lights, or may also be lights of different wavelengths (mixed lights).

In photoexposure, the light intensity is preferably within a range of from 1 mW/cm² to 50 mW/cm². When the light intensity is at least 1 mW/cm², then the producibility may increase since the photoexposure time may be reduced; and when the light intensity is at most 50 mW/cm², then it is favorable since the properties of the permanent film formed may be prevented from being degraded owing to side reaction. Also preferably, the dose in photoexposure is within a range of from 5 mJ/cm² to 1000 mJ/cm². When the dose is at least 5 mJ/cm², then the photoexposure may be free from a problem of photoexposure margin narrowing and insufficient photoexposure, and also may be free from a problem of unreacted residue adhesion to mold. On the other hand, when the dose is at most 1000 mJ/cm², then it is favorable since the composition may be prevented from being decomposed to degrade the formed permanent film.

Further, in photoexposure, the oxygen concentration in the atmosphere may be controlled to be less than 100 mg/L by introducing an inert gas such as nitrogen or argon into the system for preventing the radical polymerization from being retarded by oxygen.

Thermal curing of the composition of the invention is preferably attained at 150 to 280° C., more preferably at 200 to 250° C. The heating time is preferably from 5 to 60 minutes, more preferably from 15 to 45 minutes.

The mold material usable in the invention is described. In the photonanoimprint lithography with the composition of the invention, a light-transmissive material is selected for at least one of the mold material and/or the substrate. In the photonanoimprint lithography applied to the invention, the curable composition for nanoimprints of the invention is applied onto a substrate, and a light-transmissive mold is pressed against it, then this is irradiated with light from the back of the mold to thereby cure the curable composition for nanoimprints. Alternatively, the curable composition for photonanoimprints is applied onto a light-transmissive substrate, then a mold is pressed against it, and this is irradiated with light from the back of the mold whereby the curable composition for nanoimprints can be cured.

The photoirradiation may be attained while the mold is kept in contact with the composition or after the mold is released. In the invention, preferably, the photoirradiation is attained while the mold is kept in contact with the composition.

The mold usable in the invention has a transferable pattern formed thereon. The pattern of the mold may be formed, for example, through photolithography, electronic beam lithography or the like by which a pattern may be formed to a desired processing accuracy. In the invention, however, the mold patterning method is not specifically defined.

Not specifically defined, the light-transmissive mold material for use in the invention may be anyone having a desired strength and durability. Concretely, its examples include glass, quartz, light-transparent resin such as PMMA, polycarbonate resin or the like, transparent metal deposition film, flexible film of polydimethylsiloxane or the like, photocured film, metal film, etc.

The non-light-transmissive mold to be used in the invention where a light-transmissive substrate is used is not also specifically defined and may be any one having a predetermined strength. Concretely, examples of the mold material include ceramic material, deposition film, magnetic film, reflective film, metal material of Ni, Cu, Cr, Fe or the like, as well as SiC, silicon, silicon nitride, polysilicon, silicon oxide, amorphous silicon, etc. However, these are not limitative. Regarding the shape thereof, the mold may be any of a tabular mold or a roll mold. The roll mold is used especially when continuous production by transferring is desired.

The mold for use in the invention may be processed for surface release treatment for the purpose of enhancing the releasability of the composition of the invention from the mold. The mold of the type includes those surface-treated with a silicone-type or fluorine-containing silane coupling agent, for which, for example, commercial release agents such as Daikin's Optool DSX, Sumitomo 3M's Novec EGC-1720 and others are preferred.

In photoimprint lithography according to the production method for a cured film of the invention, in general, the mold pressure is preferably at most 10 atmospheres. When the mold pressure is at most 10 atmospheres, then the mold and the substrate are hardly deformed and the patterning accuracy tends to increase. It is also favorable since the pressure unit may be small-sized since the pressure to be given to the mold may be low. The mold pressure is preferably selected from the region capable of securing the mold transfer uniformity, within a range within which the residual film of the composition in the area of mold pattern projections may be reduced.

In the invention, the dose of photoirradiation in photoimprint lithography may be sufficiently larger than the dose necessary for curing. The dose necessary for curing may be suitably determined depending on the degree of consumption of the unsaturated bonds in the curable composition for nanoimprints and on the tackiness of the cured film as previously determined.

In the photonanoimprint lithography applied to the invention, the substrate temperature in photoirradiation may be room temperature; however, the photoirradiation may be attained under heat for enhancing the reactivity. In the former stage of photoirradiation, preferably, the system is kept in vacuum as effective for preventing contamination with bubbles or for preventing the reduction in reactivity owing to contamination with oxygen, and as effective for enhancing the adhesiveness of the curable composition for nanoimprints with mold. The system may be subjected to photoirradiation while still kept in vacuum. In the invention, the vacuum degree is preferably from 10⁻¹ Pa to ordinary pressure.

### EXAMPLES

The characteristics of the invention are described more concretely with reference to Production Examples and Examples given below. In the following Examples, the material used, its amount and the ratio, the details of the treatment and the treatment process may be suitably modified or changed not overstepping the scope of the invention. Accordingly, the invention should not be limitatively interpreted by the Examples mentioned below.

### Production Example 1

### Production of AL-1

2,2-Bis(hydroxymethyl)propionic acid (by Tokyo Chemical) (20.0 g) was dissolved in N-methylpyrrolidinone (NMP) (150 ml), and sodium hydrogencarbonate (by Wako Pure Chemical) (13.8 g) was added thereto, and then allyl bromide (by Tokyo Chemical) (19.8 g) was added thereto. This was heated up to an inner temperature of 80° C., then stirred for 8 hours, and thereafter left cooled. When the inner temperature reached lower than 35° C., NMP (50 ml) was added to it, and then 3-chloropropionyl chloride (by Tokyo Chemical) (60.6 g) was dropwise thereto. This was kept at an inner temperature of 50° C. and stirred for 2 hours, and then left cooled. When the inner temperature reached lower than 35° C., an aqueous solution of saturated sodium hydrogencarbonate (400 ml) was dropwise added to it, then ethyl acetate (400 ml) was added thereto and processed for liquid-liquid separation. The organic layer was washed with an aqueous solution of 0.2 N hydrochloric acid (250 ml). The organic layer was dried with magnesium sulfate (30 g), and the filtrate was concentrated to give a crude product of AL-1A.

Next, AL-1A was dissolved in acetonitrile (200 ml), then triethylamine (by Wako Pure Chemical) (45.3 g) was added thereto and stirred at room temperature for 4 hours, and thereafter an aqueous sodium hydrogencarbonate solution (sodium hydrogen carbonate 5 g+water 300 ml) was added thereto. Ethyl acetate (300 ml) was added to it and processed for liquid-liquid separation. The organic layer was washed with an aqueous solution of 1 N hydrochloric acid (200 ml) and saturated saline (200 ml), then the organic layer was dried with magnesium sulfate (20 g), and the filtrate was concentrated to give a crude product of AL-1.

The obtained crude product was purified through silica gel column chromatography to give AL-1 (38 g) (three-step yield, 90%).

¹H NMR and the viscosity data with an E-type viscometer of the obtained AL-1 are shown below.

¹H NMR (300 MHz, CDCl₃) δ 6.4 (d, 2H), δ 6.1 (d, 2H), δ 6.0-5.8 (m, 1H), δ 5.9 (d, 2H), δ 5.3 (dd, 2H), δ 4.6 (d, 2H), δ 4.4 (s, 4H), δ 1.3 (s, 3H)

Viscosity 14.1 mPa·s (25° C.)
Molecular weight 282.29

### Production Example 2

### Production of AL-2

In the same manner as that for AL-1 but starting from DL-malic acid (by Tokyo Chemical) (80.0 g), a crude product of AL-2 was produced. The crude product was purified through silica gel column chromatography to give AL-2 (96.2 g) (three-step yield, 60%).

¹H NMR and the viscosity data with an E-type viscometer of the obtained AL-2 are shown below.

¹H NMR (300 MHz, CDCl₃) δ 6.5 (d, 1H), δ 6.2 (dd, 1H), δ 6.0-5.8 (m, 3H), δ 5.6 (d, 1H), δ 5.4-5.2 (m, 4H), δ 4.7 (d, 2H), δ 4.6 (d, 2H)

Viscosity 13.9 mPa·s (25° C.)
Molecular weight 268.26

### Production Example 3

### Production of AL-3

In the same manner as that for AL-1 but starting from DL-tartaric acid (by Tokyo Chemical) (20.0 g), a crude product of AL-3 was produced. The crude product was purified through silica gel column chromatography to give AL-3 (32.5 g) (three-step yield, 72%).

¹H NMR and the viscosity data with an E-type viscometer of the obtained AL-3 are shown below.

¹H NMR (300 MHz, CDCl₃) δ 6.5 (d, 2H), δ 6.2 (dd, 2H), δ 6.0 (d, 2H), δ 5.9-5.7 (m, 4H), δ 5.3 (d, 4H), 5.2 (d, 4H), δ 4.8-4.6 (m, 4H)

Viscosity 76.9 mPa·s (25° C.)
Molecular weight 338.31

### Production Example 4

A commercial product of hydroxycarboxylic acid was used as a starting material and this was reacted with sodium hydrogencarbonate and allyl bromide for allyl esterification of the carboxyl group thereof. Then, this was reacted with triethylamine and acrylic acid chloride to acrylate the hydroxyl group thereof. The resulting crude product was purified through silica gel column chromatography to give AL-4 and AL-5.

### Example 1

### Preparation of Curable Composition for Photoimprints

The following polymerizable monomer AL-1 (60% by mass), the following polymerizable monomer R-1 (10% by mass), the following polymerizable monomer S-1 (30% by mass), the following photopolymerization initiator P-1 (2% by mass of the total amount of the polymerizable monomers), the following antioxidant A-1 (1% by mass of the total amount of the polymerizable monomers), the following surfactant (W-1) (0.1% by mass of the total amount of the polymerizable monomers) and the following surfactant W-2 (0.04% by mass of the total amount of the polymerizable monomers) were mixed to prepare a curable composition for photoimprints of Example 1.

### Examples 2 to 8

In the same manner as in Example 1, compositions of Examples 2 to 10 were prepared in which, however, the constitutive ingredients were changed as in Table 1 below.

The materials of the curable compositions for photoimprints are shown below.

### <(Meth)acrylate Compound>

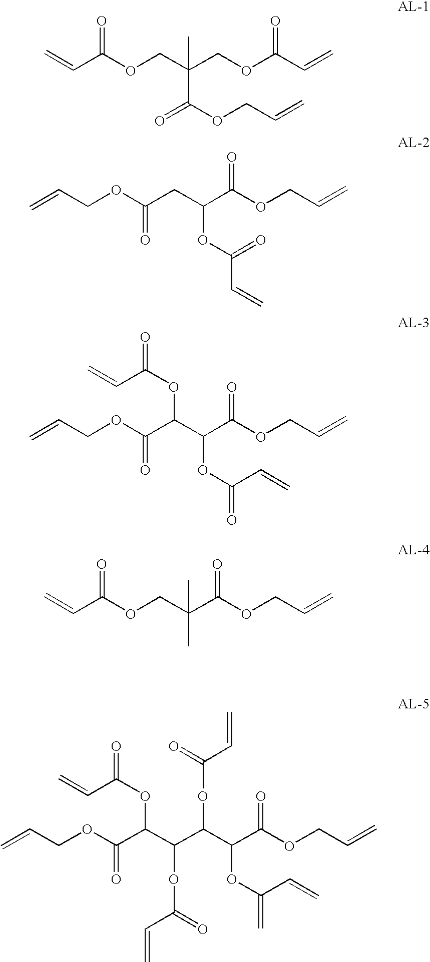

### <Monofunctional Monomer>

R-1: 3-acryloxypropyltrimethoxysilane (KBM-5103 by Shin-etsu Chemical)
R-2: benzyl acrylate (Biscoat #160, by Osaka Organic Chemical)

### <Bifunctional Monomer>

S-1: neopentyl glycol diacrylate (by Nippon Kayaku)
S-2: 1,4-butanediol diacrylate (Tokyo Chemical)

### <Trifunctional Monomer>

T-1: trimethylolpropane triacrylate (Aronix M-309, by To a Gosei)

### <Tetrafunctional Monomer>

F-1: pentaerythritol tetraacrylate (NK Ester A-TMMT, by Shin-Nakamura Chemical)
F-2: tetrafunctional urethane acrylate oligomer (U-4HA, by Shin-Nakamura Chemical)

### <Acryl Ester Monomer>

E-1: diallyl maleate (by Tokyo Chemical)

### <Photopolymerization Initiator>

P-1: 2,4,6-trimethylbenzoyl-ethoxyphenyl-phosphine oxide (Lucirin Tpo-L, by Basf)

### <Antioxidant>

### A-1: Sumilizer GA80 (by Sumitomo Chemical)

### A-2: Irganox 1035FF (by Ciba)

### <Nonionic Surfactant>

W-1: non-fluorine surfactant (Pionin D6315, by Takemoto Yushi)
W-2: fluorine-containing surfactant (Megafac F780F, by Dai-Nippon Ink Chemical)

### <Release Agent>

O-1: denatured silicone X22-3710 (by Shin-etsu Chemical)
O-2: denatured silicone KF-410 (by Shin-etsu Chemical)

### <Silane Coupling Agent>

C-1: 3-aminopropyltrimethoxysilane (KBM-903, by Shin-etsu Chemical)

TABLE 1

| | Example 1 | | Example 2 | | Example 3 | | Example 4 | | Example 5 | |
|---|---|---|---|---|---|---|---|---|---|---|
| | | added | | added | | added | | added | | added |
| | | amount, | | amount, | | amount, | | amount, | | amount, |
| | | ratio by | | ratio by | | ratio by | | ratio by | | ratio by |
| | compound | weight | compound | weight | compound | weight | compound | weight | compound | weight |
| Polymerizable | AL-1 | 60 | AL-2 | 45 | AL-3 | 25 | AL-4 | 20 | AL-5 | 25 |
| Monomer | R-1 | 10 | R-1 | 10 | R-1 | 10 | R-1 | 10 | R-1 | 20 |
| | S-1 | 30 | S-1 | 45 | R-2 | 55 | S-1 | 70 | S-1 | 55 |
| | | | | | S-1 | 10 | | | | |
| Photo- | P-1 | 2 | P-1 | 2 | P-1 | 0.5 | P-1 | 0.5 | P-1 | 0.5 |
| initiator | | | | | | | | | | |
| Antioxidant | A-1 | 1 | A-1 | 1 | A-1 | 1.5 | A-1 | 1 | A-1 | 1.5 |
| | | | A-2 | 0.5 | A-2 | 0.5 | | | A-2 | 0.5 |
| Surfactant | W-1 | 0.1 | W-1 | 0.1 | W-1 | 0.1 | W-1 | 0.1 | W-1 | 0.1 |
| | W-2 | 0.04 | W-2 | 0.04 | W-2 | 0.04 | W-2 | 0.04 | W-2 | 0.04 |

TABLE 2

| | Example 6 | | Example 7 | | Example 8 | | Example 9 | | Example 10 | |
|---|---|---|---|---|---|---|---|---|---|---|
| | | added, | | added | | added | | added | | added |
| | | amount, | | amount, | | amount, | | amount, | | amount, |
| | | ratio by | | ratio by | | ratio by | | ratio by | | ratio by |
| | compound | weight | compound | weight | compound | weight | compound | weight | compound | weight |
| Polymerizable | AL-1 | 23 | AL-1 | 23 | AL-1 | 23 | AL-1 | 23 | AL-1 | 23 |
| Monomer | R-1 | 10 | R-1 | 10 | R-1 | 10 | R-1 | 10 | R-1 | 10 |
| | S-2 | 58 | S-2 | 58 | S-2 | 58 | S-2 | 58 | S-2 | 58 |
| | F-2 | 5 | F-2 | 5 | F-2 | 5 | F-2 | 5 | F-2 | 5 |
| Silane | | | C-2 | 0.6 | | | C-2 | 0.6 | C-2 | 0.6 |
| Coupling | | | | | | | | | | |
| Agent | | | | | | | | | | |
| Photo- | P-1 | 2 | P-1 | 2 | P-1 | 2 | P-1 | 2 | P-1 | 2 |
| initiator | | | | | | | | | | |
| Antioxidant | A-1 | 1 | A-1 | 1 | A-1 | 1 | A-1 | 1 | A-1 | 1 |
| Release | O-1 | 0.3 | O-1 | 0.3 | O-1 | 0.3 | O-1 | 0.4 | O-2 | 0.4 |
| Agent | | | | | | | | | | |
| Surfactant | W-1 | 0.1 | W-1 | 0.1 | W-1 | 0.1 | | | | |
| | W-2 | 0.04 | W-2 | 0.04 | W-2 | 0.04 | | | | |

### Comparative Example 1

In the same manner as in Example 1 but using T-1 in place of AL-1, a composition of Comparative Example 1 was prepared and a resist pattern was formed.

### Comparative Example 2

In the same manner as in Example 2 but using T-1 in place of AL-2, a composition of Comparative Example 2 was prepared and a resist pattern was formed.

### Comparative Example 3

In the same manner as in Example 3 but using F-1 in place of AL-3, a composition of Comparative Example 3 was prepared and a resist pattern was formed.

### Comparative Example 4

In the same manner as in Example 1 but using T-1 in place of AL-1 and reducing the amount of T-1 to be added for keeping a low viscosity, a composition of Comparative Example 4 was prepared and a resist pattern was formed.

### Comparative Example 5

In the same manner as above but using E-1 as the polymerizable monomer, a composition of Comparative Example 5 was prepared. This was processed to form a resist pattern, however, a pattern could not be formed as the photocurability of the composition was poor.

TABLE 3

| | Comparative | | Comparative | | Comparative | | Comparative | | Comparative | |
|---|---|---|---|---|---|---|---|---|---|---|
| | Example 1 | | Example 2 | | Example 3 | | Example 4 | | Example 5 | |
| | | added | | added | | added | | added | | added |
| | | amount, | | amount, | | amount, | | amount, | | amount, |
| | | ratio by | | ratio by | | ratio by | | ratio by | | ratio by |
| | compound | weight | compound | weight | compound | weight | compound | weight | compound | weight |
| Polymerizable | T-1 | 60 | T-1 | 45 | F-1 | 25 | T-1 | 15 | E-1 | 100 |
| Monomer | R-1 | 10 | R-1 | 10 | R-1 | 10 | R-1 | 10 | | |
| | S-1 | 30 | S-1 | 45 | R-2 | 55 | S-1 | 85 | | |
| | | | | | S-1 | 10 | | | | |
| Photo- | P-1 | 2 | P-1 | 2 | P-1 | 0.5 | P-1 | 2 | P-1 | 2 |
| initiator | | | | | | | | | | |
| Antioxidant | A-1 | 1 | A-1 | 1 | A-1 | 1.5 | A-1 | 1 | A-1 | 1 |
| | | | A-2 | 0.5 | A-3 | 0.5 | | | | |
| Surfactant | W-1 | 0.1 | W-1 | 0.1 | W-1 | 0.1 | W-1 | 0.1 | W-1 | 0.1 |
| | W-2 | 0.04 | W-2 | 0.04 | W-2 | 0.04 | W-2 | 0.04 | W-2 | 0.04 |

### (Curing by Photoirradiation)

The composition comprising polymerizable monomer, photopolymerization initiator, surfactant and antioxidant as in the above Tables was prepared, then stirred and applied onto a glass substrate in a mode of spin coating so as to form thereon a layer having a thickness of 3.0 μm. The spin-coated substrate was set in an nanoimprinting apparatus with a high-pressure mercury lamp (by ORC) (lamp power, 2000 mW/cm²) as the light source. Next, a mold formed of polydimethylsiloxane (Toray Dow Corning's SILPOT 184 cured at 80° C. for 60 minutes), which had a 10-μm line/space pattern and had a groove depth of 4.0 μm, was prepared. The apparatus was vacuumed (to a vacuum degree of 10 Torr, about 1.33 kPa), the mold was fitted to the substrate, and the apparatus was purged with nitrogen (1.5 atmospheres, mold pressing pressure). This was photoexposed from the back of the mold under the condition of a lighting intensity of 10 mW/cm² and a photoexposure dose of 240 mJ/cm². After the exposure, the mold was released, and a resist pattern was thus formed.

### (Curing by Heating)

The resist pattern formed through photoexposure according to the above-mentioned method was heated in an oven at 230° C. for 30 minutes, and was thereby completely cured.

### <Evaluation of Photonanoimprint Lithography>

The compositions obtained in the above Examples and Comparative Examples were tested and evaluated according to the following evaluation methods.

### <Determination of Composition Viscosity>

The composition comprising polymerizable monomer, photopolymerization initiator, surfactant and antioxidant as in the above Tables was prepared, then stirred, and its viscosity (before curing) was measured with a rotary viscometer (Toki Sangyo's RE-80L Model) at 25±0.2° C.

The rotation speed in the measurement was 100 rpm when the viscosity was from 0.5 mPa·s to less than 5 mPa·s; 50 rpm when from 5 mPa·s to less than 10 mPa·s; 20 rpm when from 10 mPa·s to less than 30 mPa·s; 10 rpm when from 30 mPa·s to less than 60 mPa·s. Based on the viscosity range, the samples were grouped and evaluated as follows:

A: less than 12 mPa·s.
B: from 12 mPa·s to less than 18 mPa·s.
C: 18 mPa·s or more.

### <Observation of Patterning Accuracy>

The composition comprising polymerizable monomer, photopolymerization initiator, surfactant and antioxidant as in the above Tables was prepared, and applied onto a glass substrate in a mode of spin coating so as to form thereon a layer having a thickness of 3.0 μm. The spin-coated substrate was set in an nanoimprinting apparatus with a high-pressure mercury lamp (by ORC) (lamp power, 2000 mW/cm²) as the light source. A mold formed of polydimethylsiloxane (Toray Dow Corning's SILPOT 184 cured at 80° C. for 60 minutes), which had a 10-μm line/space pattern and had a groove depth of 4.0 μm, was fitted to the substrate, and this was photoexposed from the surface of the mold under the condition of 240 mJ/cm². The mold-pressing pressure was 0.8 kN, and the degree of vacuum in the apparatus during photoexposure was 10 Torr. After the exposure, the mold was released, and a resist pattern was thus formed. The resist pattern thus formed was heated in an oven at 230° C. for 30 minutes, and was thereby completely cured.

After thus transferred, the pattern profile was observed with a scanning electronic microscope of an optical microscope, and the pattern profile was evaluated as follows:

A: The transferred pattern was nearly the same as the original pattern of the mold.
B: The transferred pattern partly differed from the original pattern of the mold. (The difference was less than 10% of the original pattern.)
C: The transferred pattern partly differed from the original pattern of the mold. (The difference was from 10% to less than 20% of the original pattern.)
D: The transferred pattern clearly differed from the original pattern of the mold, or the thickness of the transferred pattern differed from that of the original pattern by at least 20%.
E: As the curability was poor in photoexposure, the subsequent process could not be attained.

### <Scratch Resistance>

The composition comprising polymerizable monomer, photopolymerization initiator, surfactant and antioxidant as in the above Tables was prepared, and applied onto a silicon wafer by spin coating. Then, using a high-pressure mercury lamp, this was irradiated with UV ray to a photoexposure dose of 240 mJ/cm², thereby forming a cured film thereon having a thickness of 3 μm. According to the method of JIS K5600-5-4, the film was tested for the pencil strength thereof and evaluated as follows:

A: pencil hardness of at least 4H.
B: pencil hardness of from 3H to 4H.
C: pencil hardness of less than 3H.
<Cured Film Adhesiveness after Photoexposure>

The composition comprising polymerizable monomer, photopolymerization initiator, surfactant and antioxidant as in the above Tables was prepared, and applied onto a silicon nitride substrate by spin coating. Then, using a high-pressure mercury lamp, this was irradiated with UV ray to a photoexposure dose of 240 mJ/cm², thereby forming a cured film thereon having a thickness of 3 μm. An adhesive tape was stuck to it, and peeled away, and the sample was visually checked as to whether or not the photocured resist pattern was stuck to the tape, and evaluated as follows. The results are shown in the following Table. The adhesive tape used herein is Nitto Denko's Cellophane Tape® No. 29.

A: No pattern adhered to the tape.
B: The pattern partly peeled and adhered to the tape.
C: The pattern was completely peeled and adhered to the tape.

TABLE 4

| | | | | | Compre- |
|---|---|---|---|---|---|
| | | Patterning | Scratch | Adhesive- | hensive |
| | Viscosity | Accuracy | Resistance | ness | Evaluation |
| Example 1 | A | A | A | A | A |
| Example 2 | A | A | A | A | A |
| Example 3 | A | A | A | A | A |
| Example 4 | A | A | B | A | B |
| Example 5 | B | B | A | A | B |
| Example 6 | A | A | A | B | B |
| Example 7 | A | A | A | A | A |
| Example 8 | A | A | A | B | B |
| Example 9 | A | A | A | A | A |
| Example 10 | A | A | A | A | A |
| Comparative | C | D | A | C | C |
| Example 1 | | | | | |
| Comparative | B | C | A | C | C |
| Example 2 | | | | | |
| Comparative | C | D | A | C | C |
| Example 3 | | | | | |
| Comparative | A | A | D | B | C |
| Example 4 | | | | | |
| Comparative | A | E | — | — | — |
| Example 5 | | | | | |

The compositions of Examples 1 to 10 all had a low viscosity and had an extremely high transfer patterning accuracy, and these all had excellent scratch resistance and adhesiveness. It is known that all these compositions are comprehensively excellent. In particular, it is known that the compositions of Examples 1 to 3 where the polymerizable monomer has three or four allyl ester groups and acrylic groups in total are especially excellent.

On the other hand, the compositions of Comparative Examples 1 to 3 had a higher viscosity than that of the compositions of the invention, and their patterning accuracy and adhesiveness were poor.

The composition of Comparative Example 4 was inferior to the compositions of the invention in point of the scratch resistance of the cured film.

The present invention has made it possible to provide a curable composition for nanoimprint lithography, which has a low viscosity and has a high transfer patterning accuracy and gives a cured film excellent in scratch resistance and adhesiveness.

While the present invention has been described in detail and with reference to specific embodiments thereof, it will be apparent to one skilled in the art that various changes and modifications can be made therein without departing from the spirit and scope thereof.

The present disclosure relates to the subject matter contained in Japanese Patent Application No. 286069/2008 filed on Nov. 7, 2008, which is expressly incorporated herein by reference in its entirety. All the publications referred to in the present specification are also expressly incorporated herein by reference in their entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims set forth below.

What is claimed is:

1. A curable composition for photoimprints, comprising a (meth)acrylate compound represented by the following formula (1): 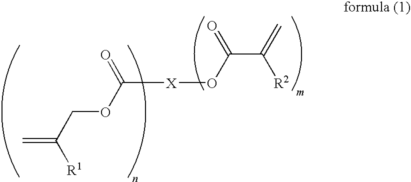
wherein R¹ and R² each represent a hydrogen atom or a methyl group; X represents a single bond or an aliphatic group; n and m each indicate an integer of 1 or more; when m or n is 2 or more, the respective R¹'s or R²'s may be the same or different.

2. The curable composition for photoimprints according to claim 1, wherein X in formula (1) is an aliphatic group having from 2 to 4 carbon atoms.

3. The curable composition for photoimprints according to claim 1, wherein n+m in formula (1) is an integer of from 3 to 5.

4. The curable composition for photoimprints according to claim 1, wherein n and m in formula (1) each are an integer of from 1 to 3.

5. The curable composition for photoimprints according to claim 1, wherein the viscosity at 25° C. of the (meth)acrylate compound of formula (1) is at most 100 mPa·s.

6. The curable composition for photoimprints according to claim 1, wherein the (meth)acrylate compound is represented by one of the following formulae AL-1 to AL-5:

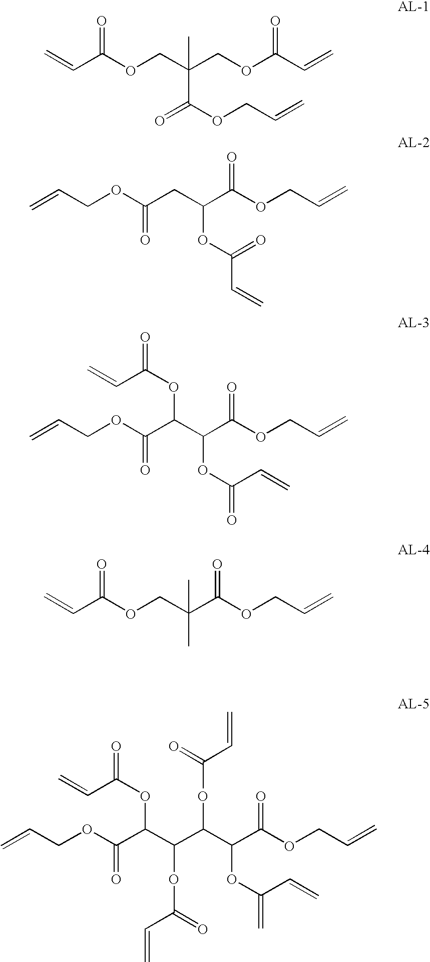

7. The curable composition for photoimprints according to claim 1, wherein the (meth)acrylate compound is represented by one of the following formulae AL-1 to AL-3:

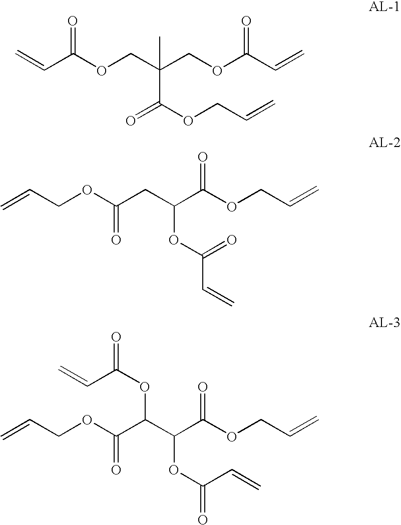

8. The curable composition for photoimprints according to claim 1, which further comprises a silicon compound.

9. The curable composition for photoimprints according to claim 1, which further comprises an antioxidant.

10. A compound represented by one of the following formulae AL-1 to AL-3:

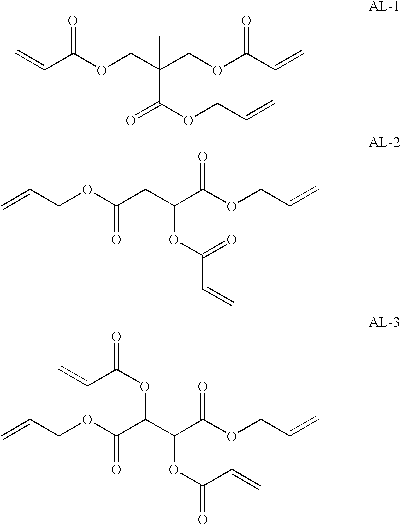

11. The compound according to claim 10 represented by the formula AL-1.

12. The compound according to claim 10 represented by the formula AL-2.

13. The compound according to claim 10 represented by the formula AL-3.

14. A cured product produced by curing a curable composition for photoimprints which comprises a (meth)acrylate compound represented by the following formula (1): 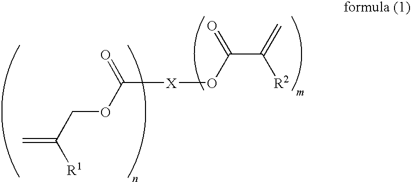
wherein R¹ and R² each represent a hydrogen atom or a methyl group; X represents a single bond or an aliphatic group; n and m each indicate an integer of 1 or more; when m or n is 2 or more, the respective R¹'s or R²'s may be the same or different.

15. A component of liquid-crystal display devices, comprising a cured product produced by curing a curable composition for photoimprints which comprises a (meth)acrylate compound represented by the following formula (1): 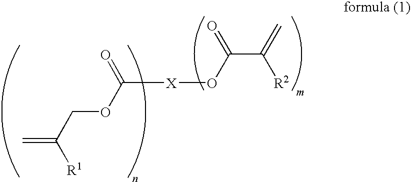
wherein R¹ and R² each represent a hydrogen atom or a methyl group; X represents a single bond or an aliphatic group; n and m each indicate an integer of 1 or more; when m or n is 2 or more, the respective R¹'s or R²'s may be the same or different.

16. A method for producing a cured product, comprising:
applying a curable composition for photoimprints onto a substrate and patterning it thereon,
pressing a mold against the patterned layer, and
photoirradiating the patterned layer,
wherein the curable composition for photoimprints comprises a (meth)acrylate compound represented by the following formula (1): 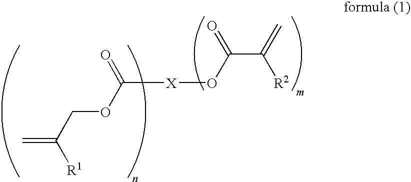
wherein R¹ and R² each represent a hydrogen atom or a methyl group; X represents a single bond or an aliphatic group; n and m each indicate an integer of 1 or more; when m or n is 2 or more, the respective R¹'s or R²'s may be the same or different.

17. The method for producing a cured product according to claim 16, which further comprises heating the photoirradiated patterned layer.
